(19) United States
(12) Patent Application Publication
(10) Pub. No.: US 2013/0183679 A1
(43) Pub. Date: Jul. 18, 2013

(54) METHOD FOR DETECTING THE PRESENCE OF BACTERIAL STRAINS RESISTANT TO ANTIBIOTICS IN A BIOLOGICAL SAMPLE

(71) Applicants: Marie-Cecile Ploy, Limoges (FR); Olivier Barraud, Limoges (FR)

(72) Inventors: Marie-Cecile Ploy, Limoges (FR); Olivier Barraud, Limoges (FR)

(21) Appl. No.: 13/821,691

(22) PCT Filed: Sep. 9, 2011
(86) PCT No.: PCT/EP11/65652

§ 371 Date: Apr. 3, 2013

Related U.S. Application Data
(60) Provisional application No. 61/381,505, filed on Sep. 10, 2010.

(51) Int. Cl. C12Q 1/68

(52) U.S. Cl. CPC C12Q 1/689
USPC 435/6.12

(57) ABSTRACT

The invention relates to the field of molecular diagnostic, in particular for the detection of the presence of gram-negative bacterial strains resistant to antibiotic in a biological sample. The invention more specifically relates to an in vitro method for detecting the presence of gram-negative bacterial strains resistant to antibiotics in a biological sample, said method comprising the steps of: a) providing a biological sample; b) preparing said biological sample for nucleic acid amplification; c) performing nucleic acid amplification using (i) nucleic acid from said biological sample as a template, (ii) at least one or more set of primers specific of bacterial genes encoding integrase of integrons of class 1, 2 and 3, and, (iii) at least one or more set of primers specific of bacterial genes encoding CTX-M type β-lactamases; and, d) determining the presence or absence of amplicons; wherein the presence of at least one amplicon is indicative of a high likelihood that said biological sample contains bacterial strains resistant to antibiotics. The method may be carried out directly on clinical samples, e.g. from septic patients.

### FIELD OF THE INVENTION

The invention relates to the field of molecular diagnostic methods, in particular for the detection of the presence of Gram-negative bacterial strains resistant to antibiotics in a biological sample. The invention more specifically relates to an in vitro method for detecting the presence of Gram-negative bacterial strains resistant to antibiotics in a biological sample, said method comprising the steps of:
a) providing a biological sample;
b) preparing said biological sample for nucleic acid amplification;
c) performing nucleic acid amplifications using (i) nucleic acid from said biological sample as a template, (ii) at least one or more set of primers specific of bacterial genes encoding integrase of integrons of class 1, 2 and 3, and (iii) at least one or more set of primers specific of bacterial genes encoding CTX-M type β-lactamases; and,
d) determining the presence or absence of amplicons resulting from the nucleic acid amplifications of step c);
wherein the presence of at least one amplicon is indicative of a high likelihood that said biological sample contains Gram-negative bacterial strains resistant to antibiotics.

The method may be carried out from bacterial isolates, from blood cultures or directly on clinical samples, e.g. from septic patients.

### BACKGROUND OF THE INVENTION

When faced with new antibiotic selection pressures, bacteria can adapt very rapidly by mutating or acquiring new genetic elements. In recent years the emergence and dissemination of resistant bacteria have been facilitated by the growing use of antibiotics. Moreover, bacteria possess a variety of highly complex genetic elements that allow the horizontal transfer of resistance genes to members of different species or even different genera. Along with transposons and plasmids, integrons play a major role in the spread of antibiotic resistance among Gram-negative bacteria [12]. They are composed of 1) a gene, intI, encoding an integrase, 2) a specific recombination site, attI, and 3) a promoter, Pc, necessary for expression of gene. Integrons capture and express resistance genes contained in so-called “gene cassettes” via integrase-mediated recombination events [13]. More than 130 different gene cassettes conferring resistance to almost all antibiotics have been described [14]. Five classes of these resistance integrons (RI) have been described, based on the sequence of the IntI1 integrase protein; classes 1, 2 and 3 being the most extensively studied [13]. Several studies have shown a tight link between integron detection and multidrug resistance (>80%), suggesting that integrons could be practical predictive markers of acquired resistance in Gram-negative bacteria [11, 12, 14]. Therefore their detection can constitute a first “screening” step for multidrug resistance in Gram-negative bacteria. In addition, sensitive methods are needed to detect RIs directly in complex genetic environments (body fluids, environmental samples, etc;), and thus dispense conventional bacterial culture. On the other hand, recently, enterobacteriaceae producing extended-spectrum β-lactamases (ESBLs), the CTX-M enzymes have emerged within the hospital and community settings as an important cause of urinary tract infection [Pitout et al., J Antimicrob Chemother 2005; 56: 52-59]. Assays for detecting CTX-M-type β-lactamases in clinical isolates have been described for example in Pitout et al., Clin Microbiol Infect 2007; 13: 291-297. Therefore, for clinicians, the choice of antibiotics is complicated by the emergence of multidrug-resistant bacteria [5], even in patients with community-acquired infections [6].

This is particularly true in infectious diseases when the early introduction of appropriate antibiotics is the key of successful treatment, i.e in sepsis where every hour without inadequate antibiotic therapy reduces the survival rate by nearly 8% [4]. Sepsis results from a systemic inflammatory response to bacterial, viral or fungal infection. The annual incidence of sepsis is estimated 50-95 cases per 100,000 inhabitants. There are approximately 750,000 cases of sepsis per year in the United States and the frequency is increasing, given an aging population with increasing numbers of patients infected with treatment-resistant organisms [1]. Sepsis is the reason for 15% of ICU admissions (75,000 per year in France) and the second cause of death in the ICU (135,000 in Europe and 215,000 per year in the USA [2]). Nine percent of patients develop severe sepsis and 3% septic shock, with a mortality rate close to 20% and over 50%, respectively. In France, the estimated frequency of septic shock is 8.2 per 100 ICU admissions (increasing from 7.0% in 1993 to 9.7% in 2000) and multidrug-resistant bacteria are increasingly isolated [3]. Conventional bacteriological investigations are not suited for early tailored therapy, as it takes 36 to 48 hours to identify the causative species and to determine its antibiotic susceptibility. Moreover, culture is negative in one-third of the sepsis cases. As a result, clinicians use to choose probabilistic antibiotic treatment based on clinical presentation and epidemiological characteristics, with a potential adjustment with the results of conventional microbiological results 48 hours later if necessary. In order to hasten the identification of the causative microorganism, molecular techniques and other non-culture-based methods have been developed [7, 8]. Most of these techniques focus on identifying the infecting organism, either from positive blood-culture samples [9] or directly from blood samples [10]. However, these techniques fail to provide antibiotic susceptibility information for Gram-negative bacteria for which antibiotic resistance is potentially mediated by hundreds of different genes, and genotypic assays cannot be used to predict resistance in routine practice. Microchip-based approaches can currently only be applied to bacterial isolates or to positive blood cultures [11], but they lack sensitivity to be applied directly to biological samples.

Therefore, there is still a need for a rapid and simple assay for detecting resistant Gram-negative bacteria in biological samples, for example in clinical samples, with a sufficient sensitivity. Ideally, this assay would be suitable for guiding clinicians in initial antibiotherapy.

The present invention provides a specific and sensitive assay combining the detection of genes encoding integrases of classes 1, 2 and 3 integrons and genes encoding CTX-M β-lactamases. One major advantage of this method is that it can be applied not only to bacterial isolates but also directly to more complexe biological samples such as clinical samples. Moreover, it can detect the majority of existing resistant strains with a good sensitivity and a minimum of sample manipulations and technical steps. Furthermore, the method of the invention is rapid and gives a result in three hours after reception of the samples in the laboratory. This method may advantageously be used for the early detection of markers of bacterial resistance in biological samples (blood and non blood), for example to predict antibiotic resistance of Gram-negative bacteria in septic patients.

### SUMMARY OF THE INVENTION

The invention relates to an in vitro method for detecting the presence of gram-negative bacterial strains resistant to antibiotics in a biological sample, said method comprising the steps of
(a) providing a biological sample;
(b) preparing said biological sample for nucleic acid amplification; preferably obtaining a biological sample from a blood culture without any DNA extraction step;
(c) performing nucleic acid amplifications using (i) nucleic acid from said biological sample as a template, (ii) at least one or more set of primers specific of bacterial genes encoding integrase of integrons of class 1, 2 and 3; and,
(d) determining the presence or absence of amplicons resulting from the nucleic acid amplifications of step c);
   wherein the presence of at least one amplicon is indicative of a high likelihood that said biological sample contains bacterial strains resistant to antibiotics.

The invention also relates to an in vitro method for detecting the presence of gram-negative bacterial strains resistant to antibiotics in a biological sample, said method comprising the steps of:
(a) providing a biological sample;
(b) preparing said biological sample for nucleic acid amplification;
(c) performing nucleic acid amplifications using (i) nucleic acid from said biological sample as a template, (ii) at least one or more set of primers specific of bacterial genes encoding integrase of integrons of class 1, 2 and 3, and (iii) at least one or more set of primers specific of bacterial genes encoding CTX-M type β-lactamases; and,
(d) determining the presence or absence of amplicons resulting from the nucleic acid amplifications of step c);
   wherein the presence of at least one amplicon is indicative of a high likelihood that said biological sample contains bacterial strains resistant to antibiotics.

In one specific embodiment, the invention relates to an in vitro method for detecting the presence of gram-negative bacterial strains resistant to antibiotics in a biological sample, said method comprising the steps of:
(a) obtaining a biological sample from a blood culture without any DNA extraction step;
(b) preparing said biological sample for nucleic acid amplification;
(c) performing nucleic acid amplifications using (i) nucleic acid from said biological sample as a template, (ii) at least one or more set of primers specific of bacterial genes encoding integrase of integrons of class 1, 2 and 3, and (iii) at least one or more set of primers specific of bacterial genes encoding CTX-M type β-lactamases; and,
(d) determining the presence or absence of amplicons resulting from the nucleic acid amplifications of step c);
   wherein the presence of at least one amplicon is indicative of a high likelihood that said biological sample contains bacterial strains resistant to antibiotics.

In one specific embodiment, said one or more sets of primers specific of bacterial genes encoding integrases of integrons of class 1, 2 and 3 essentially consist of three sets of primers, one set of primers specifically hybridizing to highly conserved regions in gene encoding integrase of class 1 integron, a second set of primers specifically hybridizing to highly conserved regions in gene encoding integrase of class 2 integron and a third set of primers specifically hybridizing to highly conserved regions in gene encoding integrase of class 3 integron.

In another specific embodiment said primers specifically hybridizing to a highly conserved region in a determined gene is a set of primers that have nucleotide sequences that are identical or have no more than 1, 2 or 3 nucleotide substitution or deletion when compared to the corresponding nucleic acid sequences in said highly conserved region to which they best aligned using a sequence alignment algorithm.

In a preferred embodiment of the method of the invention, the following three sets of primers (i)-(iii), specific of bacterial genes encoding integrase of integrons of class 1, 2 and 3, respectively, are used:
i. primers 5′IntI1 of SEQ ID NO:5 and 3′IntI1 of SEQ ID NO:6, said set of primers being specific of genes encoding integrase of class 1 integrons;
ii. primers 5′IntI2 of SEQ ID NO:7 and 3′IntI2 of SEQ ID NO:8, said set of primers being specific of genes encoding integrase of class 2 integrons; and,
iii. primers 5′IntI3 of SEQ ID NO:9 and 3′IntI3 of SEQ ID NO:10, said set of primers being specific of the genes encoding integrase of class 3 integrons;

The three sets of primers specific of bacterial genes encoding integrases of integrons of class 1, 2 and 3 may be used together in a triplex real-time PCR amplification.

In another embodiment of the method of the invention; one or more set of primers specific of CTX-M type β-lactamases are selected among those that hybridize to regions of bla_{CTXM} genes conserved between the five phylogenetic groups consisting of CTX-M-1 group, CTX-M-2 group, CTX-M-8 group, CTX-M-9 group and CTX-M-25 group. For example, said one or more set of primers specific of CTX-M type β-lactamases essentially consists of the set of primers 5′ CTXM of SEQ ID NO:11 and 3′CTXM of SEQ ID NO:12.

In one specific embodiment, at step c) of the method of the invention, one triplex real-time PCR amplification is performed on the biological sample using the following three sets of primers (i)-(iii) as defined below:
i. primers 5′IntI1 of SEQ ID NO:5 and 3′IntI1 of SEQ ID NO:6, said set of primers being specific of the gene encoding integrase of class 1 integrons;
ii. primers 5′IntI2 of SEQ ID NO:7 and 3′IntI2 of SEQ ID NO:8, said set of primers being specific of the gene encoding integrase of class 1 integrons; and,
iii. primers 5′IntI3 of SEQ ID NO:9 and 3′IntI3 of SEQ ID NO:10, said set of primers being specific of the gene encoding integrase of class 3 integrons;
   and wherein said biological sample is prepared from blood culture without DNA extraction step.

In one preferred embodiment, at step c) of the method of the invention, on the one hand, one triplex real-time PCR amplification is performed from one portion of the biological sample using the following three sets of primers (i)-(iii) as defined below:
i. primers 5′IntI1 of SEQ ID NO:5 and 3′IntI1 of SEQ ID NO:6, said set of primers being specific of the gene encoding integrase of class 1 integrons;
ii. primers 5′IntI2 of SEQ ID NO:7 and 3′IntI2 of SEQ ID NO:8, said set of primers being specific of the gene encoding integrase of class 1 integrons; and,
iii. primers 5′IntI3 of SEQ ID NO:9 and 3′IntI3 of SEQ ID NO:10, said set of primers being specific of the gene encoding integrase of class 3 integrons;
   and; on the other hand, one PCR amplification step, e.g. one simplex real-time PCR is performed from another portion of the biological sample using the following set of primers (iv):
iv. primers 5′CTXM of SEQ ID NO:11 and 3′CTXM of SEQ ID NO:12.

In preferred embodiments of the method of the invention, said biological sample is obtained from a human patient, for example a patient suffering from sepsis.

In other embodiments, said biological sample is obtained from an animal biological sample.

The invention further relates to a kit for detecting antibiotic resistance in a biological sample, comprising at least three sets of primers specific of bacterial genes encoding integrase of integrons of class 1, 2 and 3 respectively and at least one or more set of primers specific of bacterial genes encoding CTX-M type β-lactamases.

One example of a kit according to the present invention is a kit comprising the following sets of primers (i)-(iv):
i. primers 5′IntI1 of SEQ ID NO:5 and 3′IntI1 of SEQ ID NO:6, said set of primers being specific of the gene encoding integrase of class 1 integrons;
ii. primers 5′IntI2 of SEQ ID NO:7 and 3′IntI2 of SEQ ID NO:8, said set of primers being specific of the gene encoding integrase of class 2 integrons;
iii. primers 5′IntI3 of SEQ ID NO:9 and 3′IntI3 of SEQ ID NO:10, said set of primers being specific of the gene encoding integrase of class 3 integrons; and,
iv. primers 5′CTXM of SEQ ID NO:11 and 3′CTXM of SEQ ID NO:12, said set of primers being specific of the gene encoding CTX-M type β-lactamases.

The invention also relates to an in vitro diagnostic method for early diagnosis of a human patient susceptible to be in need of broad spectrum antibiotherapy, said method comprising the steps of carrying out the molecular diagnostic method of the invention as described above, wherein said biological sample is obtained from a patient presenting the clinical symptoms of bacterial infection, wherein the detection of at least one amplicon is indicative that said patient is susceptible to be in need of broad spectrum antibiotherapy.

### DETAILED DESCRIPTION OF THE INVENTION

A first object of the invention is to provide molecular diagnostic methods, and in particular an in vitro method for detecting the presence of gram-negative bacterial strains resistant to antibiotic in a biological sample, said method comprising the steps of
(a) providing a biological sample;
(b) preparing said biological sample for nucleic acid amplification; preferably the biological sample is prepared from a blood culture without any DNA extraction step,
(c) performing nucleic acid amplifications using (i) nucleic acid from said biological sample as a template, (ii) at least one or more set of primers specific of bacterial genes encoding integrase of integrons of class 1, 2 and 3; and,
(d) determining the presence or absence of amplicons resulting from the nucleic acid amplifications of step c);
   wherein the presence of at least one amplicon is indicative of a high likelihood that said biological sample contains bacterial strains resistant to antibiotics.

A related object of the invention is to provide molecular diagnostic methods, and in particular an in vitro method for detecting the presence of gram-negative bacterial strains resistant to antibiotic in a biological sample, said method comprising the steps of
(a) providing a biological sample;
(b) preparing said biological sample for nucleic acid amplification, preferably the biological sample is prepared from a blood culture without any DNA extraction step,
(c) performing nucleic acid amplifications using (i) nucleic acid from said biological sample as a template, (ii) at least one or more set of primers specific of bacterial genes encoding integrase of integrons of class 1, 2 and 3, and (iii) at least one or more set of primers specific of bacterial genes encoding CTX-M type β-lactamases; and,
(d) determining the presence or absence of amplicons resulting from the nucleic acid amplifications of step c);
   wherein the presence of at least one amplicon is indicative of a high likelihood that said biological sample contains bacterial strains resistant to antibiotics.

The method of the invention enables to identify multi-resistant strains, such as those comprising integrons of Class 1, 2 and/or 3 and/or those expressing CTX-M type beta-lactamases. Most of these strains are gram-negative bacteria, for example enterobacteriaceae, such E. coli species or Klebsiella spp.

In one embodiment, a multi-resistant strain is a strain resistant to antibiotics of different groups, preferably more than 2 antibiotic groups.

### Providing a Biological Sample

The method of the invention can be carried out on any biological sample where there is a need to determine the presence of bacterial strains resistant to antibiotics.

As used herein, the term “biological sample” refers to a sample that contains nucleic acid materials.

As used herein, the term “nucleic acid” is meant a polymeric compound comprising nucleoside or nucleoside analogs which have nitrogenous heterocyclic bases, or base analogs, linked together by nucleic acid backbone linkages (e.g., phosphodiester bonds) to form a polynucleotide. Conventional RNA and DNA are included in the term “nucleic acid” as are analogs thereof. The nucleic acid backbone may include a variety of linkages, for example, one or more of sugar-phosphodiester linkages, peptide-nucleic acid bonds, phosphorothioate or methylphosphonate linkages or mixtures of such linkages in a single oligonucleotide. Sugar moieties in the nucleic acid may be either ribose or deoxyribose, or similar compounds with known substitutions. Conventional nitrogenous bases (A, G, C, T, U), known base analogs (eg inosine), derivatives of purine or pyrimidine bases and “abasic” residues (i.e., no nitrogenous base for one or more backbone positions) are included in the term nucleic acid. That is, a nucleic acid may comprise only conventional sugars, bases and linkages found in RNA and DNA, or may include both conventional components and substitutions (e.g., conventional bases and analogs linked via a methoxy backbone, or conventional bases and one or more base analogs linked via an RNA or DNA backbone).

A biological sample as used in the methods of the invention may comprise dead or living biological organisms. In one embodiment, said sample is obtained from cultures of micro-organisms or bacterial isolates, from plants, animals, organic waste, soil samples from natural environment and the like, water sample from natural environment, such as sea, lake or rivers, dusts or air sample from natural or building environment.

In one specific embodiment, said sample is obtained from animal, for example non-human mammal. In one specific embodiment, said sample is a clinical sample obtained from human, in particular a human patient. For example, said biological sample may be obtained from urine, blood including without limitation peripheral blood or plasma, stool, sputum, bronchoalveolar fluid, endotracheal aspirates; wounds, cerebrospinal fluid, lymph node, exsudate and more generally any human biopsy tissue or body fluids, tissues or materials.

In a related embodiment, the starting material is inoculated in culture media appropriate for bacterial proliferation, e.g., Columbia blood agar plates under conditions sufficient for obtaining bacterial proliferation. For example, blood cultures is used to be cultured during 5 days according to usual protocols, e.g. using BacT/ALERT 3D system (bioMérieux, France) [Saito T, Senda K, Takakura S, Fujihara N, Kudo T, Iinuma Y, Tanimoto M, Ichiyama S. J Infect Chemother. 2003 September; 9(3):227-32]. Accordingly, in this embodiment, the biological sample that is used for the method of the invention is the cultured biological sample, e.g. a positive blood culture from a human patient, e.g from a human septic patient.

### Preparing Said Biological Sample for Nucleic Acid Amplification

At step b) of the method, the sample may first be treated to physically, chemically and/or mechanically disrupt tissue or cell structure, thus releasing intracellular components. In one specific embodiment, a DNA extraction step is carried out at step b) of the method of the invention. Such extraction step should allow obtaining nucleic acids in a good enough quality for its use as nucleic acids template for nucleic acid amplifications at step c). Extraction methods are well described in the art and any appropriate methods can be used depending on the amount of starting material, the quality of the sample and the nature of the biological material or nucleic acids contained in the biological sample.

In one specific embodiment with biological sample obtained from human patient, such as blood, urine, stool or endotracheal aspirates, the total DNA extraction method is used.

In another embodiment, no DNA extraction method is performed prior to step c).

### Performing Nucleic Acid Amplification Steps

According to the method of the invention, at least one nucleic acid amplification step is performed. This one or more amplification steps should, on the one hand, allow the detection of amplicons specific of the presence of genes encoding integrase of integrons of class 1, 2 or 3 (i.e intI1, intI2 and intI3 genes respectively) in the biological sample, and/or, on the other hand, the detection of amplicons specific of the presence of genes coding for CTX-M type β-lactamases (i.e. bla_{CTX-M}). In one specific embodiment, two amplification steps are performed in parallel on a separated portion of said biological sample, one for the integrons detection and another for the CTXM detection.

As used herein, the term “nucleic acid amplification” refers to any known procedure for obtaining multiple copies of a target nucleic acid sequence or its complementary or fragments thereof, using sequence-specific probes, referred to as primers. Known amplification methods include, for example, Polymerase Chain Reaction (PCR), Ligase Chain Reaction (LCR), Strand Displacement Amplification (SDA), replicate-mediated amplification and transcription-mediated amplification.

In one preferred embodiment, said one or more amplification steps carried out in the method of the invention are PCR amplifications. Methods for carrying out PCR amplifications are thoroughly described in the literature, for example in “PCR Primer: A laboratory Manual” Dieffenbach and Dveksler, eds. Cold Spring Harbor Laboratory Press, 1995.

In one related specific embodiment, Real-Time PCR also called quantitative PCR or qPCR is used in at least one amplification step. Real-time PCR is advantageously used to simultaneously quantify and amplify one (simplex) or more (multiplex) target nucleic acid sequences. Real-time PCR allows not only the detection of a target sequence in a biological sample but also its quantification. Real-time PCR is widely used in molecular diagnostic, in particular, for medical biology, and in microbiology [Espy et al., 2006, Clinical Microbiology Reviews, 2006, 19(1), 165-256]. The term “real-time” refers to periodic monitoring during PCR. Indeed, the real-time procedure follows the general pattern of PCR, but amplicons are quantified after each round of amplification.

Devices for performing Real-time PCR are commercially available (e.g. SmartCycler® II from Cepheid®, LightCycler® from Roche® or MX3005P® from Stratagene). For quantification of the amplicons with real-time PCR, intercalating agent, such as SYBR® Green I molecule, or other fluorescent dyes including fluorescein and rhodamine dyes may be used. Fluorogenic probes may advantageously be used especially for multiplex PCR. Examples of fluorogenic probes are the hydrolysis probes also known under the name Taqman®, or molecular beacon probe or SCORPION® probe and Fluorescence Resonance Energy Transer probes.

As used herein, the term “primers” refers to an oligonucleotide sequence of at least 10 nucleotides, for example from 10 to 50 nucleotides, for example, from 18 to 25 nucleotides, that is designed to hybridize with a complementary portion of a target sequence, and will function as the starting point for the polymerization of nucleotides (primer extension) at each amplification cycle during PCR.

In one preferred embodiment; the primers specific of integrase of integrons of class 1, 2 and 3 used in the method of the invention may have a melting temperature Tm from 59° C. to 61° C., preferably of about 60° C. (as calculated according to Chen H, Zhu G. 1997 June; 22(6):1158-60). They may preferably be selected with a GC % from 40 to 60%. If several sets of primers are used together in the same amplification step in the method of the invention, it is preferable that the primers have at least the same Tm. Such primers may preferably not hybridize to themselves or to other primers used in the same amplification step of the method. Algorithms or softwares for designing and selecting appropriate target nucleotide sequences and primers are available in the art. See for example, Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386. Source code available at http://fokker.wi.mitedu/primer3/.

As used herein “a set of primers” refers to at least two primers, one primer hybridizing to the one end of one strand of a target nucleic acid to be amplified, and the other primer hybridizing to the other strand at the other end of the target nucleotide sequence to be amplified. A set of primers thereby defines the end sequences of the amplified product or amplicon. Preferably, the set of primers specific of integrons of Class 1, 2 and 3 are defined so as to amplify target sequences below 200 bp, more preferably below 150 bp.

As used herein, the term “amplicons” refers to nucleic acids that have been synthesized (amplified) during the amplification steps, having a nucleotide sequence corresponding to the target nucleotide sequence. According to the methods of the invention, an amplicon that may be detected according to the methods of the invention is a fragment of a nucleotide sequence of a gene encoding integrase of integrons of class 1, 2 and/or 3 and/or of a gene encoding CTX-M type β-lactamases.

### Design and Molecular Characterization of the Sets of Primers Specific of Genes Encoding Integrase of Integrons of Class 1, 2 and 3

At least one set of primers used in the amplification step of the methods of the invention is specific of genes encoding integrase of integrons of class 1, 2 and 3.

As used herein, the term “genes encoding integrase of integrons of class 1, 2 and 3” refers to the bacterial genes intI1, intI2 and intI3 as defined in SEQ ID NO:1, 2 and 3 respectively.

The term “specific” means that the set of primers is designed to amplify nucleic acid fragment of genes encoding integrase of integrons of class 1, 2 or 3, without amplification of related genes, and even closely related genes, for example, genes encoding integrase of superintegrons.

In a preferred embodiment, one set of primers is designed so as to specifically amplify nucleic acid fragment from intI1 gene of SEQ ID NO:1, a second set of primers is designed so as to specifically amplify nucleic acid fragment from intI2 gene of SEQ ID NO:2, and a third set of primers is designed so as to specifically amplify nucleic acid fragment from intI3 gene of SEQ ID NO:3.

In one specific embodiment, the sets of primers used for specific amplification of bacterial genes encoding integrase of integrons of class 1, 2 and 3 essentially consist of one set of primers specifically hybridizing to highly conserved regions in gene encoding integrase of class 1 integron, a second set of primers specifically hybridizing to highly conserved regions in gene encoding integrase of class 2 integron and a third set of primers specifically hybridizing to highly conserved regions in gene encoding integrase of class 3 integron.

As used herein, the term “specifically hybridizing” means that the primer is at least 60%, 70%, 80%, 90%, 95% or 100% identical to its target nucleotide sequence.

As used herein, the percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

Algorithms such as those based on CLUSTALW computer program (Thompson Nucl Acid Res. 2 (1994), 4673-4680) may be used. Alternatively, the Geneious software available from http://www.geneious.com/, Drummond A J, Ashton B, Cheung M, Heled J, Kearse M, Moir R, Stones-Havas S, Thierer T, Wilson A (2009) Geneious v4.7, may be used.

The percent identity between two nucleotide sequences may be performed using BLAST and BLAST2.0 algortihms (Altschul, (1997) Nucl. Acids. Res. 25: 3389-3402; Altschul (1993) J. Mol. Evol. 36: 290-300; Altschul (1990) J. Mol. Biol. 215: 403-410). The BLASTN program for nucleic acid sequences uses as defaults a word length (W) of 11, an expectation (E) of 10, M=5, N=4, and a comparison of both strands.

Highly conserved region of intI1 gene can be determined by comparing all intI1 sequences available in the gene databases, for example, from GenBank, using a multiple sequence alignment algorithm such as Geneious software, and determining the most conserved region, for example, regions sharing 100% identity among all known sequences.

The same procedure can be followed for determining highly conserved region of intI2 and intI3 genes. Primers can be designed to be identical to these highly conserved regions and meeting the preferred criteria as described in the previous section.

In one embodiment, a highly conserved region in the gene intI1 encoding integrase of class 1 integron is comprised in a nucleic acid sequence ranging from position 529 to 815 of SEQ ID NO:1; a highly conserved region in a gene intI2 encoding integrase of class 2 integron is comprised in a nucleic acid sequence ranging from position 138 to 495 of SEQ ID NO:2, and a highly conserved region in the gene intI3 encoding integrase of class 3 integron is comprised in a nucleic acid sequence ranging from position 773 to 910 of SEQ ID NO:3.

In one specific embodiment, the term “specifically hybridizing to a highly conserved region in a gene” means that the set of primers is designed to have nucleotide sequences that are identical or have no more than 1, 2 or 3 nucleotide substitution or deletion when compared to the corresponding nucleotide sequences in said highly conserved region to which they best aligned using a sequence alignment algorithm such as Geneious software.

In preferred embodiments, one or more of the following set of primers may be used:
i. primers 5′IntI1 of SEQ ID NO:5 and 3′IntI1 of SEQ ID NO:6, said set of primers being specific of the intI1 encoding integrase of class 1 integrons;
ii. primers 5′IntI2 of SEQ ID NO:7 and 3′IntI2 of SEQ ID NO:8, said set of primers being specific of the intI2 encoding integrase of class 2 integrons; and,
iii. primers 5′IntI3 of SEQ ID NO:9 and 3′IntI3 of SEQ ID NO:10, said set of primers being specific of the intI3 encoding integrase of class 3 integrons.

The three sets of primers as described above may be used in distinct amplification reaction steps, performed in parallel or sequentially on aliquots of the biological sample or related nucleic acid material extracted or prepared from said biological sample. In a more preferred embodiment, the three sets of primers are advantageously used together in a single multiplex quantitative PCR. A detailed protocol of such triplex quantitative PCR has been described for example in Barraud et al. [Barraud O, Baclet M C, Denis F, Ploy M C. J Antimicrob Chemother. 2010 August; 65(8):1642-5].

Variant sets of primers as those primers (i)-(iii) described above, for example, primers containing at least 10, 15 or 20 consecutive nucleotides of the primers described above may be used, as long as they retain their capacity to amplify with substantially the same sensitivity and specificity their target nucleotide sequence as compared to the original sets of primers, when performing the same triplex quantitative PCR as described in Barraud et al. [Barraud O, Baclet M C, Denis F, Ploy M C. J Antimicrob Chemother. 2010 August; 65(8):1642-5].

Other variant sets of primers that may be used are primers that are identical to the primers (i)-(iii) as defined above, except that they have no more than 1, 2, 3, 4 or 5 nucleotides substitution, deletion and/or insertion when compared with the corresponding original primer.

### Design and Molecular Characterization of the Set of Primers Specific of CTX-M Type β-Lactamases

At least another set of primers is specific of CTX-M type β-lactamases. β-lactamases confer resistance to β-lactam drugs. These enzymes hydrolyse the β-lactam ring of antibiotics such as penicillin, cephalosporins, cephamycins, and carbanepems. The CTX-M type β-lactamases have emerged as a new type of β-lactamases, characteristic of ESBLs bacterial strains. To this day, over 85 CTX-M derivatives, classified into five phylogenetic groups consisting of CTX-M-1 group, CTX-M-2 group, CTX-M-8 group, CTX-M-9 group and CTX-M-25 group have been documented.

Examples of primers specific of CTX-M genes have been described in the Art, for example in WO 2010/096723.

In one embodiment, one or more set of primers specific of CTX-M type β-lactamases are selected among those that hybridize to regions of bla_{CTXM} genes conserved between the five phylogenetic groups consisting of CTX-M-1 group, CTX-M-2 group, CTX-M-8 group, CTX-M-9 group and CTX-M-25 group.

In one preferred embodiment, one set of primers specific of CTX-M type β-lactamases is used consisting of the primers 5′CTXM of SEQ ID NO:11 and 3′CTXM of SEQ ID NO:12. A detailed protocol for use of said CTXM primers in the method of the present invention has been described for example in Bonnet R, et al, J. Antimicrob Agents Chemother. 2001 August; 45(8):2269-75.

In one embodiment, the primers are used in real time PCR amplification for detection and quantification of genes coding CTX-M type β-lactamases.

### Detection of the Amplicons and Diagnostic

At step d) of the methods of the invention, the presence or absence of amplicons is determined. Means for detecting amplicons are well known in the art and will be selected according to the amplification method that is used. For a review, see Lazar J G. Advanced methods in PCR product detection. PCR Methods Appl. 1994 August; 4(1):S1-14 and Espy M J, et al. Real-time PCR in clinical microbiology: applications for routine laboratory testing. Clin Microbiol Rev. 2006 January; 19(1):165-256.

In one specific embodiment, for detecting amplicons, the amplicons may be visualized by running the reaction mixture obtained at step c) of the method on an electrophoresis agarose gel. The size of the amplicons can be predicted and the presence of a nucleic acid band on the gel at the predicted size as compared to a negative control sample is indicative of the presence of an amplicon. For assessing the presence of false positive, a labelled nucleic acid probes specific of the target nucleotide sequence may be hybridized to the amplicons, using hybridization procedures such as Southern Blot.

In another specific embodiment, using real-time PCR, the amount of amplicons produced during the amplification step is determined. The real-time quantification of the amplicons enables to determine the presence or absence of the amplicons, but also to quantify the amount of starting material used as a template in the biological sample. Methods for quantifying amplicons using real-time PCR will be selected according to the probes and device that will be used, as described above.

The inventors have shown that the detection of (i) nucleic acid specific of integrases of integrons of class 1, 2 or 3, and/or (ii) nucleic acid specific of CTX-M type β-lactamases is sufficient to provide a specific, sensitive and rapid diagnostic of the presence of bacterial strains resistant to antibiotics in a biological sample.

In the methods of the invention, the “presence or absence” of an amplicon may be determined by comparing the results of the amplification steps with those obtained with positive and negative controls. An example of negative control may be obtained by the use of a biological sample derived from a similar source of the test biological sample, (e.g a blood source from a healthy human as compared to blood source from a septic patient), but known not to contain any resistant bacteria. An example of positive control may be isolates of laboratory strains known to be CTX-M positive and/or integron positive. An example of negative control may be isolates of laboratory strains known to be CTX-M negative, i.e. not to express bla_{CTXM} genes, and/or IntI negative, i.e., not to express intI genes.

In one embodiment, the presence of an amplicon is determined when the amount of said amplicon is significantly higher than the amount observed with the negative control. In general, the amount of amplicon observed in the negative control should be undetectable or barely detectable.

In another embodiment, the absence of an amplicon is determined when the amount of said amplicon is undetectable or detectable with amounts not significantly higher than the amount of amplicon observed with the negative control.

### Kits for Detecting Resistant Bacterial Strain in a Biological Sample

It is another aspect of the invention to provide a kit for carrying out the molecular diagnostic methods as described above.

The kit may comprise at least the primers specific of genes encoding integrase of integrons of Class 1, 2 and 3 and the primers specific of genes encoding CTX-M type β-lactamase.

In one embodiment, the kit comprises at least one or more sets of primers selected from the group consisting of
i. primers 5′IntI1 of SEQ ID NO:5 and 3′IntI1 of SEQ ID NO:6, said set of primers being specific of the gene encoding integrase of class 1 integrons;
ii. primers 5′IntI2 of SEQ ID NO:7 and 3′IntI2 of SEQ ID NO:8, said set of primers being specific of the gene encoding integrase of class 2 integrons;
iii. primers 5′IntI3 of SEQ ID NO:9 and 3′IntI3 of SEQ ID NO:10, said set of primers being specific of the gene encoding integrase of class 3 integrons; and,
iv. primers 5′CTXM of SEQ ID NO:11 and 3′CTXM of SEQ ID NO:12, said set of primers being specific of the gene encoding CTX-M type β-lactamases.

In one preferred embodiment, the kit comprises the following four sets of primers
i. primers 5′IntI1 of SEQ ID NO:5 and 3′IntI1 of SEQ ID NO:6, said set of primers being specific of the gene encoding integrase of class 1 integrons;
ii. primers 5′IntI2 of SEQ ID NO:7 and 3′IntI2 of SEQ ID NO:8, said set of primers being specific of the gene encoding integrase of class 2 integrons;
iii. primers 5′IntI3 of SEQ ID NO:9 and 3′IntI3 of SEQ ID NO:10, said set of primers being specific of the gene encoding integrase of class 3 integrons; and,
iv. primers 5′CTXM of SEQ ID NO:11 and 3′CTXM of SEQ ID NO:12, said set of primers being specific of the gene encoding CTX-M type β-lactamases.

The kit may further comprise buffers and reagents suitable for the preparation of the biological sample and/or nucleic acid amplification steps and/or detection of the amplicons.

In one specific embodiment, the kit may further comprise typical reagents used in PCR reaction such as, DNA polymerases, deoxyribonucleoside triphosphates (dNTPs, ie dATP, dCTP, dTTP, dGTP), an aqueous buffer medium that may include monovalent ions, e.g. potassium chloride, a source of divalent cations, e.g. magnesiumn, and a buffering agent such as TRIS, HEPES or MOPS and the like. Other agents that may be present in the buffer medium include chelating agents such as EDTA and/or BSA. The kit may further comprise dyes and other probes useful for real-time or qPCR. The kit may also contain control DNA template for positive and negative control. The kit may also comprise appropriate instructions for use.

The kit may be presented in a carrier being compartmentalized to receive one or more containers such as tubes or vials.

### Applications of the Methods of the Invention for Monitoring and Predicting Antibiotherapy in Patients

The methods of the invention may be applied to all fields of molecular diagnostic where there is a need to detect the presence of antibiotic resistant organism in a biological sample.

In preferred related embodiments, the method of the invention is used for quick determination of the presence of bacterial strains resistant to antibiotics, from bacterial isolates from positive blood cultures or directly from clinical samples from human patients, e.g. suffering from sepsis.

Therefore, it is another aspect of the present invention to provide an in vitro diagnostic method for early diagnosis of a human patient susceptible to be in need of broad spectrum antibiotherapy, comprising the steps of the method for determining the presence of bacterial strains resistant to antibiotics, as described above, wherein said biological sample is obtained from a patient presenting the clinical symptoms of bacterial infection, wherein the detection of at least one amplicon is indicative that said patient is susceptible to be in need of broad spectrum antibiotherapy.

In one embodiment, a patient presenting the clinical symptoms of bacterial infection is a sepsis patient, in particular of abdominal or urinary tract origin.

The invention will be further illustrated by the following figures and examples. However, these examples and figures should not be interpreted in any way as limiting the scope of the present invention.

### Example

### 1. Detecting Multi-Resistant Bacterial Strains Directly from Clinical Sample or Positive Blood Cultures

Conventional bacteriology involves first the inoculation of a biological sample on appropriate culture media so as to grow bacteria eventually present in the sample. Identification and antibiotic susceptibility testing (antibiogram) are secondarily performed on bacterial isolates. This methodology used in all microbiology laboratories is based on the bacterial culture and requires a minimum of 36 hours to obtain the final results (18-24 hours for bacterial growth followed by 10 hours for identification and antibiotic susceptibility). This period is sometimes extended by several days cause the speed of growth of bacteria and/or of the presence of several bacteria, which requires subculture steps.

The problem of this methodology, which nevertheless remains the only one giving a complete antibiotic susceptibility pattern, is that the clinician has to introduce a probabilistic antibiotic therapy awaiting the final result. His only assistance pending the antibiogram is the result of the direct examination of the sample: the Gram strain can guide the therapeutic choice, without guaranteeing the antibiotic susceptibility of the germ in question.

In order to provide faster answers, molecular biology techniques have recently been developed. Most of these techniques are applied on isolated bacterial cultures, but due to their low sensitivity, they cannot be used directly on biological samples. Moreover, they primarily focus on bacterial identification but few are interested in antibiotic resistance, especially for Gram-negative bacteria due to technical difficulties in detecting hundreds of genes involved. Faced with these few tools available, we propose to detect directly from a biological sample (serum, urine, ascites, bile, respiratory samples . . . ) the presence of integrons. Indeed, integrons are bacterial genetic elements involved in acquired antibiotic resistance encountered primarily in Gram-negative bacteria and considered as markers of acquired resistance to antibiotics.

1.1 DNA Extraction from Clinical Samples

1.1.1 DNA Extraction from Clinical Samples Except Blood Cultures

To implement the method of detection of the invention in biological samples, as described in paragraph 1 above, the total DNA of the sample may be extracted. The technical protocol is the following; it differs depending on the nature of the biological sample:
Sample fluids such as whole blood, urine, CSF, bile, ascites fluid, bronchoalveolar aspirates etc. . . . are extracted automatically using the easyMAG system (bioMérieux, Marcy-l'Étoile, France) or manually using the QIAamp® DNA Mini Kit (Qiagen, Courtaboeuf, France).
Respiratory specimens other than BAL (sputum, tracheal aspirations, bronchial aspirations, . . . ) and viscous or solid samples have to undergo a prior mechanical treatment using the FastPrep® instrument.

The estimated technical time for DNA extraction is of the order of 1 to 1.5 hours for liquid samples, and about 2 hours if mechanical lysis is needed.

1.1.2 No DNA Extraction is Required from Positive Blood Cultures

Normally, when a clinician wants to know if a patient has a bacteriemia, he prescribes blood cultures. It consists of a collection of whole blood inoculated directly at the bedside in flasks containing a culture medium. The vials are then incubated in a system, which regularly measures CO₂ production by bacteria. When it reaches a threshold, the controller emits a signal and considers the blood culture as “positive”. The microbiologist then removes a few drops of the bottle, performs a Gram stain and informs the clinician. According to the staining, antibiotic susceptibility testing is directly performed; the clinician will have the results on next day. The present invention is able to look for integrons directly from blood culture bottles positive for Gram-negative bacilli, without DNA extraction step: a rapid dilution of 1:100 of the vial in sterile distilled water is enough to apply the triplex PCR. We can therefore advantageously “save” up to 24 hours compared to conventional methods and thus help the clinician to choose its initial antibiotic therapy.

1.2 Detection of Integrons of Class 1, 2 or 3 Using Triplex Ragman® PCR Amplification

Triplex PCR technique is applied to DNA extract or to positive blood cultures dilution as described (Barraud, JAC, 2010). Assays are performed with a run time of 1.5 h.

The entire methodology should therefore require an average of 3-4 hours for biological samples and less than 2 h for positive blood cultures.

### 2. Detection of CTX-M Type Beta-Lactamase Expressing Strains Using Simplex SYBR®Green Amplification

Real-time CTX-M PCR is applied directly to strain suspensions or positive blood culture dilutions, without DNA extraction step, using SYBR®Green (Takara®). PCR program is 95° C. 5 minutes followed by 40 cycles with 3 steps: 95° C. for 15 s, 55° C. for 20 s and 72° C. 40 s. Expected fusion point is 90.5° C. Assays are performed with a run time of 1.5 h. The entire methodology should therefore require less than 2 h for strains or positive blood cultures.

### 3. Testing Specificity and Sensitivity of the Method According to the Invention

3.1 Material & Methods

### Study on 120 Strains of ESBL-Producing E. Coli

We selected 120 strains of ESBL-producing E. coli isolated from various clinical samples at the Limoges Teaching hospital. These strains were selected for their phenotypic expression of an ESBL, i.e. synergy between disks of clavulanate and a third-generation cephalosprin.

Study on 148 Strains of Enterobacteriaceae Isolated from Clinical Samples.

We selected different strains belonging to different species of Enterobacteriaceae. We selected 5 groups of strains according to their phenotypic resistance to β-lactams: 1) wild-type, i.e. strains with no acquired resistance to βlactams; 2) low-level penicillinase; 3) high-level penicillinase; 4) high-level AmpC; 5) ESBL. In the group 1, according to the species, some strains were fully susceptible to βlactams or produced low-level penicillinase or low-level cephalosprinase.

### Detection Method of CTX-M and Integrons According to the Invention.

We performed bacterial DNA extraction by boiling. CTX-M detection was performed with primers 5′CTX-M of SEQ ID NO:11 and 3′CTX-M of SEQ ID NO:12, as described previously (Bonnet R, Dutour C, Sampaio J L, Chanal C, Sirot D, Labia R, De Champs C, Sirot J. Antimicrob Agents Chemother. 2001 August; 45(8):2269-75.; J. D. D. Pitout, N. Hamilton, D. L. Church, P. Nordmann and L. Poirel. Clin Microbiol Infect 2007; 13: 291-297]) and integrons detection was performed by a triplex quantitative PCR method as described by Barraud et al [Barraud O, Baclet M C, Denis F, Ploy M C. J Antimicrob Chemother. 2010 August; 65(8):1642-5].

3.2 Results

Among the 120 ESBL producing strains that were tested according to the method of the invention, 71% are intI+ (most are class 1 and a few strains are class 2) and 69% are CTX-M+. The results show that a large majority of the strains (96%) are either intI+ or CTX-M+. Therefore, the combination of both integrons and CTX-M markers appear to be suitable and sufficient for a quick, specific and sensitive detection method of multi-resistant strains in a biological sample.

The distribution of the strains according to the PCR results is described in the Table 1 below:

TABLE 1

| Absolute number of strains | | |
|---|---|---|
| (proportion %) | PCR integron | PCR CTX-M |
| 5 (4%) | − | − |
| 53 (44%) | + | + |
| 32 (26%) | + | − |
| 30 (25%) | − | + |

We then studied 148 strains with phenotypic resistance to β-lactams. The table 2 below shows the distribution of the strains according to the PCR results and depending on the phenotype.

TABLE 2

| Strain phenotypes | Integron+ | CTX-M+ |
|---|---|---|
| Wild-type (N = 30) | 2 | 0 |
| Low level penicillinase (N = 28) | 13 | 0 |
| High level penicillinase (N = 30) | 19 | 0 |
| Derepressed cephalosporinase (N = 30) | 10 | 1 |
| ESBL phenotype (N = 30) | 23 | 15 |

### 4. Detection of Integrons in Blood Cultures from Patients

Hospitalized subjects with at least 1 positive blood cultures (Bact-Alert®, 5 days incubation) with Gram-negative rods (GNB). Two hundred and five (205) patients have been included in the final study.

4.1 Positive Blood Cultures

As shown in Table 3, 158 out of the 205 blood cultures contained only one species and 47 grew at least 2 bacterial species.

TABLE 3
Blood cultures, Enterobacteriaceae (E), Non Enterobacteriaceae (NE)
including P. aeruginosa, Haemophilus, Acinetobacter, Gram
positive bacteria (GP), Anaerobic (A), Yeasts (Y) results

| | E | NE | A | | | | |
|---|---|---|---|---|---|---|---|
| Only 1 isolate | 124 | 23 | 11 | | | | |
| n = 158 | | | | | | | |
| | | E + | E + | E + | E + | E + | NE + |
| | ≧2 E | GP | A | NE | NE + A | Y | GP |
| Polymicrobial | 18 | 17 | 4 | 3 | 1 | 1 | 3 |
| (at least 2 | | | | | | | |
| species) | | | | | | | |
| n = 47 | | | | | | | |

4.2 Bacterial Isolates

235 Gram-negative rods were isolated (Table 3) with the following distribution:
105 E. coli strains
87 Enterobacteriaceae (except E. coli)
23 P. aeruginosa
12 Ana
8 other.

Enterobacteriaceae were the majority and E. coli the most frequently isolated species.

4.3 Integron Detection

4.3.1 Integron Detection in Gram-Negative Strains Isolated from Blood Cultures

The detection oh the 3 main classes of integrons was performed by the multiplex quantitative PCR technique (15) with the Stratagene Mx3005P machine. No class 3 integron was detected. Class 1 or 2 integrons were detected in bacteria in 53 patients, ie 25.9% of patients were [symbol] integron+ [symbol] (intI+) (Table 4).

TABLE 4
Results of integron detection for each bacterial group

| | Gram- | | | | | | |
|---|---|---|---|---|---|---|---|
| | negative | | E (except | | | | |
| | bacteria N | E | E. coli) | E. coli | P. aeruginosa | Anaerobes | Others |
| intl1+ | 53 | 49 | 14 | 35 | 4 | 0 | 0 |
| intl2+ | 7 | 7 | 2 | 5 | 0 | 0 | 0 |
| intl1+, intl2+ | 3 | 3 | 1 | 2 | 0 | 0 | 0 |
| Total intl+ | 57 | 53 | 15 | 38 | 4 | 0 | 0 |
| % intl+ | 24.3% | 27.6% | 17.2% | 36.2% | 17.4% | 0.0% | 0.0% |
| intl− | 178 | 139 | 72 | 67 | 19 | 12 | 8 |
| Total strains | 235 | 192 | 87 | 105 | 23 | 12 | 8 |

4.3.2 Integron Detection Directly from Blood Cultures

Integron detection directly from positive blood cultures without a DNA extraction step was performed with the same qPCR technique with the Smart Cycler II V2.0 machine. We tested 2 dilutions for each bottle: 1/100 et 1/1000. 5 μl of each dilution were used for the qPCR. Results are summarized in table 5.

TABLE 5
Table of concordance of integron detection between
blood cultures and bacterial isolates.

| | Triplex qPCR on blood cultures | | | | |
|---|---|---|---|---|---|
| | | | | IntI1+, | |
| | | IntI1+ | IntI2+ | IntI2+ | IntI− |
| Triplex qPCR on | IntI1+ | 49 | | | 2 |
| gram negative | IntI2+ | | 7 | | |
| isolates | IntI+, IntI2+ | | | 3 | |
| | IntI− | 1 | | | 143 |

We obtained a better concordance with the 1/100 dilution, with a 98.5% concordance rate. 2 unconformities were found for 3 samples (1.5% of cases):
qPCR intI1+ on blood culture, qPCR intI− from the bacteria (1 case): the recultivation of the bottle has to show a fifth gram-negative bacteria carrying integron that was not originally isolated
qPCR intI− on blood culture, qPCR intI1+ from the bacteria (2 cases): this discrepancy can be explained a priori by the presence of inhibitors (to be confirmed). DNA extraction from the bottle might solve this problem.

### 5. Detection of CTX-M by qPCR

The CTX-M PCR (SybrGreen) (Brasme et al, JAC, 2008) was performed with the Stratagene Mx3005P. This PCR was positive for 5 blood cultures (Table 6).

Concerning the Gram-negative bacteria, 9 expressed an ESBL phenotype and 5 out of 9 were positive for the CTX-M PCR. These 5 strains were isolated from the 5 positive blood cultures. For the remaining 4 strains, the Ct or Tm were out of the threshold chosen. (Table 6).

TABLE 6
Results of the CTX-M PCRs performed on ESBL strains and positive blood cultures.

| | PCR CTX-M (Stratagène), kit SYBRGreen | | | | | | | |
|---|---|---|---|---|---|---|---|---|
| ESBL strain | Ct strains | Tm strains | Interpretation | Ct sample | Tm sample | Interpretation | Sequencing | intl1 |
| K. pneumoniae | 17.59 | 90.80 | + | 19.27 | 90.65 | + | CTX-M 3 | + |
| E. coli | 18.87 | 90.78 | + | 22.45 | 90.59 | + | ND | + |
| K. pneumoniae | 22.30 | 90.83 | + | 25.70 | 90.58 | + | ND | + |
| K. pneumoniae | 21.97 | 90.84 | + | 27.90 | 90.61 | + | ND | + |
| Pantoea | 33.01 | 92.38 | − | 37.66 | 92.08 | − | | + |
| K. oxytoca | — | 67.75 | − | — | 86.45 | − | | + |
| E. coli | 18.03 | 90.88 | + | 23.22 | 91.00 | + | CTX-M 3 | − |
| E. cloacae | 36.30 | 92.28 | − | — | 92.03 | − | | + |
| E. coli | 29.88 | 92.34 | [symbol] | — | — | [symbol] | [symbol] | + |

PCR CTX-M strains: Tm = 90.8 +/− 0.1
PCR CTX-M sample: 90.75 +/− 0.30 [symbol]
indicates data missing or illegible when filed

Strain E. coli H205 contained a CTX-M gene (variant CTX-M 14 confirmed by sequencing) that was not detected by with the qPCR used.

Among the ESBL Enterobacteriaceae strains, 8 out of 9 were IntI+, 5 out of 9 were ctx-M+ and 4 out of 9 were intI+ and CTX-M+.

### 6. Summary Results

The following Tables 7 to 9 summarize the results of the combined detection of integron and CTX-M and the correlation with acquired multiple resistance to more than 2 antibiotic families.

TABLE 7
Results of integron and CTX-M detection
among Enterobacteriaceae strains

| Absolute number of | | |
|---|---|---|
| Enterobacteriaceae strains | | |
| (N = 192) (proportion %) | PCR integron | PCR CTX-M |
| 138 (72%) | − | − |
| 4 (2%) | + | + |
| 49 (25.5%) | + | − |
| 1 (0.5%) | − | + |

TABLE 8
Results of integron and CTX-M detection among patients

| Absolute number of | | |
|---|---|---|
| patients (N = 205) | | |
| (proportion %) | PCR integron | PCR CTX-M |
| 151 (73.5%) | − | − |
| 4 (2%) | + | + |
| 49 (24%) | + | − |
| 1 (0.5%) | − | + |

TABLE 9
Integron and CTX-M detection according to antibiotic
resistance to at least 2 antibiotic families.

| Enterobacteri- | | Acquired | Acquired | |
|---|---|---|---|---|
| aceae strains | | resistance <2 | resistance ≧2 | |
| n = 192 | | antibiotic families | antibiotic families | |
| intI+ | CTX-M+ | 0 (0%) | 4 (2.1%) | 4 (2.1%) |
| intI+ | CTX-M− | 9 (4.7%) | 40 (20.8%) | 49 (25.5%) |
| intI− | CTX-M+ | 0 (0%) | 1 (0.5%) | 1 (0.5%) |
| intI− | CTX-M− | 127 (66.2%) | 11 (5.7%) | 138 (71.9%) |
| | | 136 (70.8%) | 56 (29.2%) | 192 |

### 7. Useful Nucleotide Sequences for Practicing the Method of the Invention

TABLE 10:

| NO: | Description | Sequence |
|---|---|---|
| 1 | Gene Intl1 | ATGAAAACCGCCACTGCGCCGTTACCAC |
| | AM234698 | CGCTGCGTTCGGTCAAGGTTCTGGACCA |
| | | GTTGCGTGAGCGCATACGCTACTTGCAT |
| | | TACAGCTTACGAACCGAACAGGCTTATG |
| | | TCCACTGGGTTCGTGCCTTCATCCGTTT |
| | | CCACGGTGTGCGTCACCCGGCAACCTTG |
| | | GGCAGCAGCGAAGTCGAGGCATTTCTGT |
| | | CCTGGCTGGCGAACGAGCGCAAGGTTTC |
| | | GGTCTCCACGCATCGTCAGGCATTGGCG |
| | | GCCTTGCTGTTCTTCTACGGCAAGGTGC |
| | | TGTGCACGGATCTGCCCTGGCTTCAGGA |
| | | GATCGGAAGACCTCGGCCGTCGCGGCGC |
| | | TTGCCGGTGGTGCTGACCCCGGATGAAG |
| | | TGGTTCGCATCCTCGGTTTTCTGGAAGG |
| | | CGAGCATCGTTTGTTCGCCCAGCTTCTG |
| | | TATGGAACGGGCATGCGGATCAGTGAGG |
| | | GTTTGCAACTGCGGGTCAAGGATCTGGA |
| | | TTTCGATCACGGCACGATCATCGTGCGG |
| | | GAGGGCAAGGGCTCCAAGGATCGGGCCT |
| | | TGATGTTACCCGAGAGCTTGGCACCCAG |
| | | CCTGCGCGAGCAGCTGTCGCGTGCACGG |
| | | GCATGGTGGCTGAAGGACCAGGCCGAGG |
| | | GCCGCAGCGGCGTTGCGCTTCCCGACGC |
| | | CCTTGAGCGGAAGTATCCGCGCGCCGGG |
| | | CATTCCTGGCCGTGGTTCTGGGTTTTTG |
| | | CGCAGCACACGCATTCGACCGATCCACG |
| | | GAGCGGTGTCGTGCGTCGCCATCACATG |
| | | TATGACCAGACCTTTCAGCGCGCCTTCA |
| | | AACGTGCCGTAGAACAAGCAGGCATCAC |
| | | GAAGCCCGCCACACCGCACACCCTCCGC |
| | | CACTCGTTCGCGACGGCCTTGCTCCGCA |
| | | GCGGTTACGACATTCGAACCGTGCAGGA |
| | | TCTGCTCGGCCATTCCGACGTCTCTACG |
| | | ACGATGATTTACACGCATGTGCTGAAAG |
| | | TTGGCGGTGCCGGAGTGCGCTCACCGCT |
| | | TGATGCGCTGCCGCCCCTCACTAGTGAG |
| | | AGGTAG |
| 2 | Gene Intl2 | ATGTCTAACAGTCCATTTTTAAATTCTA |
| | AJ001816 | TACGCACGGATATGCGACAAAAAGGTTA |
| | | TGCGCTGAAAACTGAAAAAACTTACCTG |
| | | CACTGGATTAAGCGTTTTATTCTGTTTC |
| | | ACAAAAAACGTCATCCTCAGACCATGGG |
| | | CAGTGAAGAGGTCAGGCTGTTTTTATCC |
| | | AGCTTAGCAAACAGCAGACATGTAGCCA |
| | | TAAACACGCAGAAAATCGCTTTAAATGC |
| | | CCTAGCTTTTTTGTACAACAGGTTTTTA |
| | | CAACAGCCGTTGGGCGATATTGATTATA |
| | | TCCCTGCAAGCAAGCCTAGACGGCTACC |
| | | CTCTGTTATCTCTGCAAATGAAGTGCAA |
| | | CGCATTTTGCAGGTTATGGATACTCGCA |
| | | ACCAAGTTATTTTTACGCTGCTGTATGG |
| | | TGCAGGTTTGCGCATTAATGAATGCTTG |
| | | CGTTTGCGGGTTAAAGATTTTGATTTTG |
| | | ATAATGGCTGCATCACTGTGCATGACGG |
| | | TAAGGGTGGGAAAAGCAGAAACAGCCTA |
| | | CTGCCCACGCGCCTAATCCCAGCAATAA |
| | | AATAACTCATTGAGCAAGCGCGGCTTAT |
| | | TCAGCAAGACGACAACTTACAAGGCGTA |
| | | GGGCCATCGCTGCCTTTTGCTTTAGATC |
| | | ACAAATACCCTTCTGCTTATCGACAAGC |
| | | GGCGTGGATGTTTGTCTTTCCCTCCAGC |
| | | ACGCTCTGCAACCACCCGTATAACGGCA |
| | | AATTATGCCGCCATCATCTGCATGACTC |
| | | CGTTGCGCGAAAGGCATTGAAGGCAGCC |
| | | GTACAAAAAGCAGGCATCGTTAGCAAGC |
| | | GTGTCACTTGTCATACATTTCGTCACTC |
| | | GTTTGCTACGCATCTATTACAAGCGGGG |
| | | CGTGATATTCGCACTGTGCAAGAACTCT |
| | | TAGGGCATAACGATGTTAAGACCACGCA |
| | | AATCTATACGCATGTGTTGGGTCAGCAT |
| | | TTTGCCGGCACCACCAGTCCTGCGGATG |
| | | GACTGATGCTACTTATCAATCAGTAA |
| 3 | Gene IntI3 | ATGAACAGGTATAACGGATCTGCCAAAC |
| | AF416297 | CTGACTGGGTCCCTCCCCGGTCCATCAA |
| | | GCTGCTCGATCAGGTACGCGAACGGGTT |
| | | CGCTACCTGCATTACAGCCTACAGACCG |
| | | AGAAGGCTTATGTCTACTGGGCCAAGGC |
| | | ATTTGTGTTGTGGACGGCCCGCAGCCAT |
| | | GGTGGGTTTCGACATCCGCGCGAAATGG |
| | | GGCAAGCTGAAGTCGAGGGTTTTCTGAC |
| | | CATGCTCGCCACCGAGAAGCAAGTGGCG |
| | | CCGGCCACCCACCGGCAGGCGCTCAACG |
| | | CGCTGTTGTTCTTGTATCGGCAGGTGCT |
| | | GGGCATGGAATTGCCGTGGATGCAGCAG |
| | | ATTGGTCGGCCGCCAGAACGCAAGCGGA |
| | | TTCCGGTGGTGCTGACGGTGCAGGAGGT |
| | | TCAGACGTTGCTTTCGCACATGGCGGGC |
| | | ACCGAAGCGCTGTTGGCCGCCCTGCTTT |
| | | ACGGCAGTGGGTTGCGCCTGCGCGAAGC |
| | | GCTGGGCCTGCGGGTCAAGGATGTGGAT |
| | | TTCGACCGCCACGCGATCATTGTGCGCA |
| | | GCGGCAAGGGCGACAAGGACCGCGTGGT |
| | | GATGCTGCCCAGGGCGCTCGTACCTCGG |
| | | TTGCGGGCGCAGCTGATTCAGGTCCGCG |
| | | CTGTGTGGGGGCAGGACCGTGCCACGGG |
| | | GCGCGGAGGCGTGTATCTGCCTCATGCA |
| | | CTGGAGCGCAAGTACCCCAGGGCGGGCG |
| | | AGAGCTGGGCCTGGTTCTGGGTGTTTCC |
| | | ATCGGCCAAGCTGTCTGTGGACCCACAA |
| | | ACCGGCGTTGAGCGCCGCCACCACTTGT |
| | | TTGAGGAAAGACTGAACCGGCAACTAAA |
| | | AAAAGCGGTAGTTCAGGCTGGCATTGCC |
| | | AAACACGTATCTGTCCACACCCTGCGCC |
| | | ACTCATTCGCCACCCACTTGCTGCAAGC |
| | | AGGCACAGACATCCGAACGGTGCAAGAG |
| | | TTGTTGGGGCATTCGGACGTGAGCACGA |
| | | CGATGATCTACACGCATGTGCTGAAAGT |
| | | CGCTGCCGGAGGCACCTCCAGCCCGCTG |
| | | GACGCCTTGGCCTTGCACTTGTCGCCCG |
| | | GCTGA |
| 4 | Gene | ATGGTTAAAAAATCACTGCGTCAGTTCA |
| | bla_{CTX-MGQ274927} | CGCTGATGGCGACGGCAACCGTCACGCT |
| | | GTTGTTAGGAAGTGTGCCGCTGTATGCG |
| | | CAAACGGCGGACGTACAGCAAAAACTTG |
| | | CCGAATTAGAGCGGCAGTCGGGAGGAAG |
| | | ACTGGGTGTGGCATTGATTAACACAGCA |
| | | GATAATTCGCAAATACTTTATCGTGCTG |
| | | ATGAGCGCTTTGCGATGTGCAGCACCAG |
| | | TAAAGTGATGGCCGTGGCCGCGGTGCTG |
| | | GAAGAAAAGTGAAAGCGAACCAATCTGT |
| | | TAAATCAGCGAGTTGAGATCAAAAAATC |
| | | TGACTTGGTTAACTATAATCCGATTGCG |
| | | GAAAAGCACGTCGATGGGACGATGTCAC |
| | | TGGCTGAGCTTAGCGCGGCCGCGCTACA |
| | | GTACAGCGATAACGTGGCGATGAATAAG |
| | | CTGATTTCTCACGTTGGCGGCCCGGCTA |
| | | GCGTCACCGCGTTCGCCCGACAGCTGGG |
| | | AGACGAAACGTTCCGTCTCGACCGTACC |
| | | GAGCCGACGTTAAACACCGCCATTCCGG |
| | | GCGATCCGCGTGATACCACTTCACCTCG |
| | | GGCAATGGCGCAAACTCTGCGTAATCTG |
| | | ACGCTGGGTAAAGCATTGGGTGACAGCC |
| | | AACGGGCGCAGCTGGTGACATGGATGAA |
| | | AGGCAATACCACCGGTGCAGCGAGCATT |
| | | CAGGCTGGACTGCCTGCTTCCTGGGTTG |
| | | TGGGGGATAAAACCGGCAGCGGTGACTA |
| | | TGGCACCACCAACGATATCGCGGTGATC |
| | | TGGCCAAAAGATCGTGCGCCGCTGATTC |
| | | TGGTCACTTACTTCACCCAGCCTCAACC |
| | | TAAGGCAGAAAGCCGTCGCGATGTATTA |
| | | GCGTCGGCGGCTAAAATCGTCACCAACG |
| | | GTTTGTAA |
| 5 | Primer | GCCTTGATGTTACCCGAGAG |
| | 5′intI1 | |
| 6 | Primer | GATCGGTCGAATGCGTGT |
| | 3′intI1 | |
| 7 | Primer | GACGGCTACCCTCTGTTATCTC |
| | 5′intI2 | |
| 8 | Primer | TGCTTTTCCCACCCTTACC |
| | 3′intI2 | |
| 9 | Primer | GCCACCACTTGTTTGAGGA |
| | 5′intI3 | |
| 10 | Primer | GGATGTCTGTGCCTGCTTG |
| | 3′intI3 | |
| 11 | Primer | CGCTTTGCGATGTGCAG |
| | 5′CTX-M | |
| 12 | Primer | ACCGCGATATCGTTGGT |
| | 3′CTX-M | |

### REFERENCES

1. Russell J A. Management of sepsis. N Engl J Med 2006; 355(16): 1699-713.
2. Angus D C, et al. Epidemiology of severe sepsis in the United States: analysis of incidence, outcome, and associated costs of care. Crit. Care Med 2001; 29(7): 1303-10.
3. Annane D, et al. Current epidemiology of septic shock: the CUB-Rea Network. Am J Respir Crit. Care Med 2003; 168(2): 165-72.
4. Kumar A, et al. Duration of hypotension before initiation of effective antimicrobial therapy is the critical determinant of survival in human septic shock. Crit. Care Med 2006; 34(6): 1589-96.
5. Sengstock D M, et al. Multidrug-resistant Acinetobacter baumannii: an emerging pathogen among older adults in community hospitals and nursing homes. Clin Infect Dis 2010; 50(12): 1611-6.
6. Pitout J D, et al. Extended-spectrum beta-lactamase-producing Enterobacteriaceae: an emerging public-health concern. Lancet Infect Dis 2008; 8(3): 159-66.
7. Mancini N, et al. The era of molecular and other non-culture-based methods in diagnosis of sepsis. Clin Microbiol Rev 2010; 23(1): 235-51.
8. Ecker D J, et al. New technology for rapid molecular diagnosis of bloodstream infections. Expert Rev Mol Diagn 2010; 10(4): 399-415.
9. Tissari P, et al. Accurate and rapid identification of bacterial species from positive blood cultures with a DNA-based microarray platform: an observational study. Lancet 2010; 375(9710): 224-30.
10. Bloos F, et al. A multicenter trial to compare blood culture with polymerase chain reaction in severe human sepsis. Intensive Care Med 2010; 36(2): 241-7.
11. Cleven B E, et al. Identification and characterization of bacterial pathogens causing bloodstream infections by DNA microarray. J Clin Microbiol 2006; 44(7): 2389-97.
12. Leverstein-van Hall M A, et al. Multidrug resistance among Enterobacteriaceae is strongly associated with the presence of integrons and is independent of species or isolate origin. J Infect Dis 2003; 187(2): 251-9.
13. Mazel D. Integrons: agents of bacterial evolution. Nat Rev Microbiol 2006; 4(8): 608-20.
14. Partridge S R, et al. Gene cassettes and cassette arrays in mobile resistance integrons. FEMS Microbiol Rev 2009; 33(4): 757-84.
15. Barraud O, et al. Quantitative multiplex real-time PCR for detecting class 1, 2 and 3 integrons. J Antimicrob Chemother 2010; Epub, ahead of print.
16. Fluit A C, et al. Molecular detection of antimicrobial resistance. Clin Microbiol Rev 2001; 14(4): 836-71, table of contents.
17. Dillon B, et al. Multiplex PCR for screening of integrons in bacterial lysates. J Microbiol Methods 2005; 62(2): 221-32.
18. Bone R C, et al. Definitions for sepsis and organ failure and guidelines for the use of innovative therapies in sepsis. The ACCP/SCCM Consensus Conference Committee. American College of Chest Physicians/Society of Critical Care Medicine. Chest 1992; 101(6): 1644-55.
19. Dellinger R P, et al. Surviving Sepsis Campaign: international guidelines for management of severe sepsis and septic shock: 2008. Crit. Care Med 2008; 36(1): 296-327.
20. Flack V F, et al. Sample size determinations for the two rater kappa statistic Sample size determinations for the two rater kappa statistic. Psychometrika 1988; 53(3): 321-5.
21. Giraudeau B et al. Planning a reproducibility study: how many subjects and how many replicates per subject for an expected width of the 95 percent confidence interval of the intraclass correlation coefficient. Stat Med 2001; 20(21): 3205-14.
22. Walsh T R. Combinatorial genetic evolution of multiresistance. Curr Opin Microbiol 2006; 9(5): 476-82.
23. Stralin K, et al. Evaluation of a multiplex PCR for bacterial pathogens applied to bronchoalveolar lavage. Eur Respir J 2006; 28(3): 568-75.
24. Daikos G L, et al. Enterobacteriaceae bloodstream infections: presence of integrons, risk factors, and outcome. Antimicrob Agents Chemother 2007; 51(7): 2366-72.
25. Westh H, et al. Multiplex real-time PCR and blood culture for identification of bloodstream pathogens in patients with suspected sepsis. Clin Microbiol Infect 2009; 15(6): 544-51.

Throughout this application, various references describe the state of the art to which this invention pertains. The disclosures of these references are hereby incorporated by reference into the present disclosure.

1. An in vitro method for detecting the presence of gram-negative bacterial strains resistant to antibiotics in a biological sample, said method comprising:
a) providing a biological sample;
b) preparing said biological sample for nucleic acid amplification;
c) performing nucleic acid amplifications using (i) nucleic acid from said biological sample as a template, (ii) at least one or more sets of primers specific for bacterial genes encoding integrase of integrons of class 1, 2 and 3, and (iii) at least one or more sets of primers specific of bacterial genes encoding CTX-M type β-lactamases; and,
d) determining the presence or absence of amplicons resulting from the nucleic acid amplifications of step c);
wherein the presence of at least one amplicon is indicative of a high likelihood that said biological sample contains gram-negative bacterial strains resistant to antibiotics.

2. The method of claim 1, wherein said one or more sets of primers specific for bacterial genes encoding integrase of integrons of class 1, 2 and 3 comprise three sets of primers, one set of primers specifically hybridizing to highly conserved regions in a gene encoding integrase of class 1 integrons, a second set of primers specifically hybridizing to highly conserved regions in a gene encoding integrase of class 2 integrons and a third set of primers specifically hybridizing to highly conserved regions in a gene encoding integrase of class 3 integrons.

3. The method of claim 2, wherein said highly conserved region in a gene encoding integrase of class 1 integrons is comprised in a nucleic acid sequence ranging from position 529 to 815 of SEQ ID NO:1; said highly conserved region in the gene encoding integrase of class 2 integrons is comprised in a nucleic acid sequence ranging from position 138 to 495 of SEQ ID NO:2, and said highly conserved region in the gene encoding integrase of class 3 integrons. is comprised in a nucleic acid sequence ranging from position 773 to 910 of SEQ ID NO:3.

4. The method of claim 2, wherein primers specifically hybridizing to a highly conserved region in a determined gene are a set of primers that have nucleotide sequences that are identical or have no more than 1, 2 or 3 nucleotide substitutions or deletions when compared to corresponding nucleotide sequences in said highly conserved region of said determined gene to which they best align using a sequence alignment algorithm.

5. The method according to claim 1, wherein the following three sets of primers (i)-(iii), specific for bacterial genes encoding integrase of integrons of class 1, 2 and 3, respectively, are used:
i) primers 5′IntI1 of SEQ ID NO:5 and 3′IntI1 of SEQ ID NO:6, said set of primers being specific for the gene encoding integrase of class 1 integrons;
ii) primers 5′intI2 of SEQ ID NO:7 and 3′IntI2 of SEQ ID NO:8, said set of primers being specific for the gene encoding integrase of class 2 integrons; and,
iii) primers 5′IntI3 of SEQ ID NO:9 and 3′IntI3 of SEQ ID NO:10, said set of primers being specific for the gene encoding integrase of class 3 integrons.

6. The method according to claim 5, wherein the three sets of primers specific for bacterial genes encoding integrase of integrons of class 1, 2 and 3 are used together in a triplex real-time PCR amplification.

7. The method according to claim 1, wherein said one or more sets of primers specific for CTX-M type β-lactamases are selected among those that hybridize to regions of bla_{CTXM} genes conserved between the five phylogenetic groups consisting of CTX-M-1 group, CTX-M-2 group, CTX-M-8 group, CTX-M-9 group and CTX-M-25 group.

8. The method according to claim 1, wherein said one or more set of primers specific for CTX-M type β-lactamases comprise the primers 5′CTXM of SEQ ID NO:11 and 3′CTXM of SEQ ID NO:12.

9. The method according to claim 1, wherein at step c), one triplex real-time PCR amplification is performed from one part of the biological sample, using the following three sets of primers (i)-(iii):
i. primers 5′IntI1 of SEQ ID NO:5 and 3′IntI1 of SEQ ID NO:6, said set of primers being specific for the gene encoding integrase of class 1 integrons;
ii. primers 5′IntI2 of SEQ ID NO:7 and 3′IntI2 of SEQ ID NO:8, said set of primers being specific for the gene encoding integrase of class 2 integrons; and,
iii. primers 5′IntI3 of SEQ ID NO:9 and 3′IntI3 of SEQ ID NO:10, said set of primers being specific for the gene encoding integrase of class 3 integrons;
and one amplification step is performed from another part of the biological sample using the following set of primers (iv):
iv. primers 5′CTXM of SEQ ID NO:11 and 3′CTXM of SEQ ID NO:12.

10. The method according to claim 1, wherein said biological sample is obtained from a human patient.

11. The method of according to claim 9, wherein said biological sample is a blood sample from a human patient.

12. The method according to claim 1, wherein said biological sample is obtained from an animal biological sample.

13. A kit for detecting antibiotic resistant bacterial strains in a biological sample, comprising at least three sets of primers specific of bacterial genes encoding integrase of integrons of class 1, 2 and 3 and at least one or more sets of primers specific for bacterial genes encoding CTX-M type β-lactamases.

14. The kit of claim 13, comprising the following sets of primers
i. primers 5′IntI1 of SEQ ID NO:5 and 3′IntI1 of SEQ ID NO:6, said set of primers specific for the gene encoding integrase of class 1 integrons;
ii. primers 5′IntI2 of SEQ ID NO:7 and 3′IntI2 of SEQ ID NO:8, said set of primers specific for the gene encoding integrase of class 2 integrons;
iii. primers 5′IntI3 of SEQ ID NO:9 and 3′IntI3 of SEQ ID NO:10, said set of primers specific for the gene encoding integrase of class 3 integrons; and,
iv. one set of primers specific for the gene encoding CTX-M type β-lactamases.

15. An in vitro diagnostic method for early diagnosis of a human patient susceptible to be in need of broad spectrum antibiotherapy, said method comprising,
a) providing a biological sample;
b) preparing said biological sample for nucleic acid amplification;
c) performing nucleic acid amplifications using (i) nucleic acid from said biological sample as a template, (ii) at least one or more sets of primers specific for bacterial genes encoding integrase of integrons of class 1, 2 and 3, and (iii) at least one or more sets of primers specific of bacterial genes encoding CTX-M type β-lactamases; and,
d) determining the presence or absence of amplicons resulting from the nucleic acid amplifications of step c);
wherein said biological sample is obtained from a patient presenting the clinical symptoms of bacterial infection, and wherein the detection of at least one amplicon is indicative that said patient is in need of broad spectrum antibiotherapy.

16. The method of claim 10, wherein said human patient is suffering from sepsis.


SEQUENCE LISTING (12 sequences)

SEQ ID NO: 1 - DNA, 1014 bp, Salmonella enterica
atgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagctgtcgcgtgcacgggcatggtggctgaaggaccaggccgagggccgcagcggcgttgcgcttcccgacgcccttgagcggaagtatccgcgcgccgggcattcctggccgtggttctgggtttttgcgcagcacacgcattcgaccgatccacggagcggtgtcgtgcgtcgccatcacatgtatgaccagacctttcagcgcgccttcaaacgtgccgtagaacaagcaggcatcacgaagcccgccacaccgcacaccctccgccactcgttcgcgacggccttgctccgcagcggttacgacattcgaaccgtgcaggatctgctcggccattccgacgtctctacgacgatgatttacacgcatgtgctgaaagttggcggtgccggagtgcgctcaccgcttgatgcgctgccgcccctcactagtgagaggtag

SEQ ID NO: 2 - DNA, 978 bp, Escherichia coli
atgtctaacagtccatttttaaattctatacgcacggatatgcgacaaaaaggttatgcgctgaaaactgaaaaaacttacctgcactggattaagcgttttattctgtttcacaaaaaacgtcatcctcagaccatgggcagtgaagaggtcaggctgtttttatccagcttagcaaacagcagacatgtagccataaacacgcagaaaatcgctttaaatgccctagcttttttgtacaacaggtttttacaacagccgttgggcgatattgattatatccctgcaagcaagcctagacggctaccctctgttatctctgcaaatgaagtgcaacgcattttgcaggttatggatactcgcaaccaagttatttttacgctgctgtatggtgcaggtttgcgcattaatgaatgcttgcgtttgcgggttaaagattttgattttgataatggctgcatcactgtgcatgacggtaagggtgggaaaagcagaaacagcctactgcccacgcgcctaatcccagcaataaaataactcattgagcaagcgcggcttattcagcaagacgacaacttacaaggcgtagggccatcgctgccttttgctttagatcacaaatacccttctgcttatcgacaagcggcgtggatgtttgtctttccctccagcacgctctgcaaccacccgtataacggcaaattatgccgccatcatctgcatgactccgttgcgcgaaaggcattgaaggcagccgtacaaaaagcaggcatcgttagcaagcgtgtcacttgtcatacatttcgtcactcgtttgctacgcatctattacaagcggggcgtgatattcgcactgtgcaagaactcttagggcataacgatgttaagaccacgcaaatctatacgcatgtgttgggtcagcattttgccggcaccaccagtcctgcggatggactgatgctacttatcaatcagtaa

SEQ ID NO: 3 - DNA, 1041 bp, Serratia marcescens
atgaacaggtataacggatctgccaaacctgactgggtccctccccggtccatcaagctgctcgatcaggtacgcgaacgggttcgctacctgcattacagcctacagaccgagaaggcttatgtctactgggccaaggcatttgtgttgtggacggcccgcagccatggtgggtttcgacatccgcgcgaaatggggcaagctgaagtcgagggttttctgaccatgctcgccaccgagaagcaagtggcgccggccacccaccggcaggcgctcaacgcgctgttgttcttgtatcggcaggtgctgggcatggaattgccgtggatgcagcagattggtcggccgccagaacgcaagcggattccggtggtgctgacggtgcaggaggttcagacgttgctttcgcacatggcgggcaccgaagcgctgttggccgccctgctttacggcagtgggttgcgcctgcgcgaagcgctgggcctgcgggtcaaggatgtggatttcgaccgccacgcgatcattgtgcgcagcggcaagggcgacaaggaccgcgtggtgatgctgcccagggcgctcgtacctcggttgcgggcgcagctgattcaggtccgcgctgtgtgggggcaggaccgtgccacggggcgcggaggcgtgtatctgcctcatgcactggagcgcaagtaccccagggcgggcgagagctgggcctggttctgggtgtttccatcggccaagctgtctgtggacccacaaaccggcgttgagcgccgccaccacttgtttgaggaaagactgaaccggcaactaaaaaaagcggtagttcaggctggcattgccaaacacgtatctgtccacaccctgcgccactcattcgccacccacttgctgcaagcaggcacagacatccgaacggtgcaagagttgttggggcattcggacgtgagcacgacgatgatctacacgcatgtgctgaaagtcgctgccggaggcacctccagcccgctggacgccttggccttgcacttgtcgcccggctga

SEQ ID NO: 4 - DNA, 876 bp, Escherichia coli
atggttaaaaaatcactgcgtcagttcacgctgatggcgacggcaaccgtcacgctgttgttaggaagtgtgccgctgtatgcgcaaacggcggacgtacagcaaaaacttgccgaattagagcggcagtcgggaggaagactgggtgtggcattgattaacacagcagataattcgcaaatactttatcgtgctgatgagcgctttgcgatgtgcagcaccagtaaagtgatggccgtggccgcggtgctgaagaaaagtgaaagcgaaccgaatctgttaaatcagcgagttgagatcaaaaaatctgacttggttaactataatccgattgcggaaaagcacgtcgatgggacgatgtcactggctgagcttagcgcggccgcgctacagtacagcgataacgtggcgatgaataagctgatttctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcgtaatctgacgctgggtaaagcattgggtgacagccaacgggcgcagctggtgacatggatgaaaggcaataccaccggtgcagcgagcattcaggctggactgcctgcttcctgggttgtgggggataaaaccggcagcggtgactatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccaacggtttgtaa

SEQ ID NO: 5 - DNA, 20 bp, Artificial Sequence
  Synthetic primer
gccttgatgttacccgagag

SEQ ID NO: 6 - DNA, 18 bp, Artificial Sequence
  Synthetic primer
gatcggtcgaatgcgtgt

SEQ ID NO: 7 - DNA, 22 bp, Artificial Sequence
  Synthetic primer
gacggctaccctctgttatctc

SEQ ID NO: 8 - DNA, 19 bp, Artificial Sequence
  Synthetic primer
tgcttttcccacccttacc

SEQ ID NO: 9 - DNA, 19 bp, Artificial Sequence
  Synthetic primer
gccaccacttgtttgagga

SEQ ID NO: 10 - DNA, 19 bp, Artificial Sequence
  Synthetic primer
ggatgtctgtgcctgcttg

SEQ ID NO: 11 - DNA, 17 bp, Artificial Sequence
  Synthetic primer
cgctttgcgatgtgcag

SEQ ID NO: 12 - DNA, 17 bp, Artificial Sequence
  Synthetic primer
accgcgatatcgttggt